(19) United States
(12) Patent Application Publication
(10) Pub. No.: US 2024/0065974 A1
(43) Pub. Date: Feb. 29, 2024

(54) ORAL THIN FILM

(71) Applicant: LTS Lohmann Therapie-Systeme AG, Andemach (DE)

(72) Inventors: Christoph SCHMITZ, Rheinbrohl (DE); Marius BAUER, Andernach (DE); Michael LINN, Waldböckelheim (DE)

(21) Appl. No.: 18/272,391

(22) PCT Filed: Jan. 14, 2022
(86) PCT No.: PCT/EP2022/050786

§ 371 Date: Jul. 14, 2023

(30) Foreign Application Priority Data
Jan. 15, 2021 (DE) 10 2021 100 752.6

(51) Int. Cl. A61K 9/00; A61K 47/32; A61K 47/36

(52) U.S. Cl. CPC A61K 9/006; A61K 47/32; A61K 47/36

(57) ABSTRACT

The present invention relates to an oral thin film comprising a polymer matrix in the form of a solidified foam having voids, wherein the polymer matrix contains at least one active pharmaceutical ingredient, at least one first polymer which comprises a water-soluble polymer and at least one second polymer which comprises a mucoadhesive polymer having a number-average molecular weight of equal to or greater than 100,000 g/mol, a method for producing the oral thin film, and the use of such an oral thin film as a medicament.

The present invention relates to an oral thin film, to a method for production thereof, and to the use of such an oral thin film as a medicament.

Oral thin films are thin films containing at least one active pharmaceutical ingredient that are placed directly in the oral cavity or against the oral mucosa and dissolve or macerate there and in so doing deliver the active ingredient. These films are, especially, thin, one- or multi-layer, active-ingredient-containing polymer-based films which, when applied to a mucous membrane, especially the oral mucosa, can deliver the active ingredient directly into same. The very good blood supply to the oral mucosa ensures a rapid transfer of the active ingredient into the bloodstream. This dosage system has the advantage that the active ingredient is absorbed for the most part by the mucous membrane, thus avoiding the first-pass effect, which occurs in the case of the conventional dosage form of an active ingredient in tablet form. The active ingredient may be dissolved, emulsified or dispersed in the film.

Oral thin films in the form of a foam may have, in principle, a high loading of active pharmaceutical ingredient. These highly loaded oral thin films in the form of a foam generally demonstrate a very quick disintegration time, which is advantageous for many applications.

In other cases, however, it may be desirable that the highly loaded oral thin films in the form of a foam remain for longer in the oral cavity, for example in order to provide the active pharmaceutical ingredient for longer for transmucosal absorption.

A typical problem in the preparation of the active pharmaceutical ingredient of slowly releasing oral thin films is that the polymers typically used for this purpose form highly viscous aqueous solutions and thus reduce the solids fraction of an active ingredient solution, which, amongst other things, significantly extends the drying time.

The problem addressed by the present invention was to provide an oral thin film comprising a polymer matrix in the form of a solidified foam having voids, the disintegration time of which can be controlled as broadly as possible. In addition, an aim is to produce the oral thin film as easily and quickly as possible. Especially, an objective is to prevent highly viscous solutions and long drying times.

This aim has been addressed surprisingly by an oral thin film according to claim 1. Such an oral thin film is characterized in that it comprises a polymer matrix in the form of a solidified foam having voids, wherein the polymer matrix contains at least one active pharmaceutical ingredient, at least one first polymer which comprises a water-soluble polymer and at least one second polymer which comprises a mucoadhesive polymer having a number-average molecular weight of equal to or greater than 100,000 g/mol.

By adding a mucoadhesive polymer having a number-average molecular weight of equal to or greater than 100,000 g/mol, a foam formulation can be provided that has a longer residence time in the oral cavity. In addition, by adding a mucoadhesive polymer having a number-average molecular weight of equal to or greater than 100,000 g/mol, an increased mucoadhesivity is achieved, so that the foam better adheres to the mucosa and the active ingredient can hold the active ingredient at the absorption site.

The oral thin film can be produced in principle similarly to the previously known oral thin films, since the added mucoadhesive polymer having a number-average molecular weight of equal to or greater than 100,000 g/mol has only a small influence on the physical behaviour during the slurrying and drying. Since time-delayed dissolution may occur already from a very small quantity of mucoadhesive polymer with a number-average molecular weight of equal to or greater than 100,000 g/mol, the possible active ingredient loading is hardly affected at all.

In the present document, the word “comprising” can also mean “consisting of”.

Water-soluble polymers comprise chemically very different natural or synthetic polymers, the common feature of which is their solubility in water or aqueous media. A precondition is that these polymers have a number of hydrophilic groups sufficient for the water solubility and are not crosslinked. The hydrophilic groups may be non-ionic, anionic, cationic and/or zwitterionic.

Water-soluble is preferably understood to mean a solubility in water of greater than 100 g/L at 25° C.

The mucoadhesion process is influenced primarily by properties of the used polymers, such as degree of crosslinking, chain length and different functional groups in the polymer structure. Mucoadhesive systems are used in many mucosa-covered organelles to deliver active ingredients with local or systemic effects.

Mucoadhesion can be defined as a phenomenon of the molecular forces of attraction at the interfaces between the surfaces of the mucosa, here the oral mucosa, and the mucoadhesive polymers, which allows the polymers to adhere to the mucosa for a longer period of time.

The second polymer which comprises at least one mucoadhesive polymer has a number-average molecular weight of equal to or greater than 100,000 g/mol, preferably of greater than 200,000 g/mol or of greater than 300,000 g/mol or of greater than 400,000 g/mol or of greater than 500,000 g/mol or of greater than 600,000 g/mol or of greater than 700,000 g/mol or of greater than 800,000 g/mol or of greater than 900,000 g/mol.

Mucoadhesive polymers having a number-average molecular weight of equal to or greater than 1,000,000 g/mol or 2,000,000 g/mol or 3,000,000 g/mol or 4,000,000 g/mol or 5,000,000 g/mol or 6,000,000 g/mol or 7,000,000 g/mol are also especially preferred.

The molecular weight is determined by methods known to a person skilled in the art, such as gel permeation chromatography.

The second polymer which comprises the at least one mucoadhesive polymer having a number-average molecular weight of equal to or greater than 100,000 g/mol is preferably a water-soluble polymer as defined above.

The oral thin film according to the invention is also preferably characterised in that the first polymer comprises a polymer having a number-average molecular weight of less than 100,000 g/mol, preferably less than 80,000 or less than 60,000.

The molecular weight is determined by methods known to a person skilled in the art, such as gel permeation chromatography.

The first polymer which comprises a water-soluble polymer is preferably selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums, wherein polyvinyl alcohols are especially preferred.

The oral thin film according to the invention is also preferably characterised in that the second polymer which comprises a mucoadhesive polymer having a number-average molecular weight of equal to or greater than 100,000 g/mol, is selected from the group consisting of polyethylene oxide polymers, starch derivatives, polyacrylic acids, amylopectins and/or polyvinyl alcohols, especially mixtures of polyacrylates or polyacrylic acids and amylopectins.

Suitable second polymers are, for example polyethylene oxide polymers, such as polyethylene glycols, having a number-average molecular weight of approximately 1,000,000 g/mol or approximately 7,000,000 g/mol, which are obtainable for example under the trade names “Polyox WSR N12” or “Polyox WSR 303”.

Other suitable second polymers are mixtures of polyacrylate or polyacrylic acids and amylopectin, which are obtainable under the trade name “Proloc 15”.

Long-chain polyvinyl alcohols are also suitable as second polymer. For example, a polyvinyl alcohol having a number-average molecular weight of approximately 200,000 g/mol, which is obtainable for example under the trade name “PVA 40-88”.

The oral thin film according to the invention is also preferably characterised in that the first polymer is contained in the oral thin film in an amount of from 40 to 95 wt. %, preferably of from 45 to 85, and especially preferably of from 48 to 80, in relation to the total weight of the oral thin film.

The second polymer is present in the oral thin film in an amount of from 0.1 to 20 wt. %, preferably of from 2 to 18, and especially preferably of from 5 to 15, in relation to the total weight of the oral thin film.

The at least one active pharmaceutical ingredient is in principle not subject to any restriction, but is preferably selected from all active pharmaceutical ingredients which are suitable for oral and/or transmucosal application.

Preferred active ingredients are selected from the group comprising the active ingredient classes of analgesics, hormones, hypnotics, sedatives, antiepiletics, analeptics, psychoneurotropic drugs, neuro-muscle blockers, antspasmodics, antihistamines, antiallergics, cardiotonics, antiarrhythmics, diuretics, hypotensives, vasopressors, antidepressants, antitussives, expectorants, thyroid hormones, sexual hormones, antidiabetics, antitumour active ingredients, antibiotics, chemotherapeutics and narcotics, although this group is not exhaustive.

The at least one active pharmaceutical ingredient is especially preferably ketamine.

Ketamine is understood to mean (S)-(±)-2-(2-chlorophenyl)-2-(methylamino)cyclohexan-1-one, (R)-(±)-2-(2-chlorophenyl)-2-(methylamino)cyclohexan-1-one, and the racemate (RS)-(±)-2-(2-chlorophenyl)-2-(methylamino)cyclohexan-1-one.

(S)-ketamine is especially preferably present as a single stereoisomer of ketamine, since the analgesic and anaesthetic potency of (S)-ketamine is approximately three times higher than that of the (R) form.

The active ingredient content in the oral thin film can vary within relatively wide limits. A range of from 10 to 60 wt. %, in relation to the total weight (dry total weight) of the oral thin film, can be stated as suitable. In one embodiment, the proportion of active ingredient in the oral thin film lies rather in the lower range, for example if the active ingredient has a strong unpleasant taste, which has to be compensated for by a greater amount of taste-masking agents. In this case, a range of from 10 to 40 wt. % can be stated as suitable active ingredient fraction. In another embodiment, the proportion of active ingredient in the dosage form according to the invention lies rather in the upper range, wherein a content of from 40 to 60 wt. % and especially a content of from 45 to 55 wt. % can be stated as being especially preferred.

The active ingredient content per dosing unit is up to 100 mg, preferably up to 50 mg, especially preferably up to 40 mg, and most preferably up to 35 mg. On the other hand, the minimum active ingredient content per dosing unit should preferably be 5 mg, more preferably 10 mg, and most preferably 15 mg. Depending on the application, the active ingredient content may also lie in the upper range of the above values, for example in the range of from 50 to 100 mg. A material content of from 30 to 50 mg may also be suitable.

The oral thin film according to the invention is also preferably characterised in that the oral thin film comprises at least one auxiliary substance selected from the group comprising colouring agents, flavourings, sweeteners, plasticisers, taste-masking agents, emulsifiers, enhancers, pH regulators, humectants, preservatives and/or antioxidants.

Each of these auxiliary substances is preferably contained in an amount of from 0.1 to 40 wt. %, preferably from 0.1 to 30 wt. %, especially preferably from 0.1 to 15 wt. %, very especially preferably from 0.1 to 10 wt. %, or 0.1 to 5 wt. %, in relation to the total weight of the oral thin film.

The multi-layer oral thin film according to the invention is, in principle, not limited in the number of layers contained.

For example, embodiments are also conceivable in which the oral thin film according to the invention comprises several active-ingredient-containing layers.

The oral thin film according to the invention is characterised in that it is present in the form of a solidified foam that has voids.

An advantage of this embodiment is that, despite the comparatively high area density, faster drying can be achieved by formulating as a foam than with a comparable non-foamed composition.

The oral thin film according to the invention is preferably characterised in that the voids are isolated from one another and are preferably present in the form of bubbles, the voids being filled with air or a gas, preferably with an inert gas, especially preferably with nitrogen, carbon dioxide, helium or a mixture of at least two of these gases.

According to another embodiment, it is provided that the voids are connected to one another, preferably by forming a continuous channel system penetrating the matrix.

Said voids preferably have a volume fraction of from 5 to 98%, preferably from 50 to 80%, in relation to the total volume of the matrix layer. In this way, the advantageous effect of accelerating the dissolution of the thin film is influenced favourably.

Furthermore, surface-active substances or surfactants can be added to the oral thin film for foam formation or to the obtained foam before or after the drying in order to improve the stability of the foam before or after the drying.

Another parameter that influences the properties of the oral thin film according to the invention is the diameter of the voids or bubbles. The bubbles or voids are preferably created with the aid of a foam whipping machine, with which the diameter of the bubbles can be adjusted in a wide range, almost arbitrarily. The diameter of the bubbles or voids can thus lie in the range of 0.01 to 60 μm. The diameter especially preferably lies in the range of 10 and 50 μm.

The oral thin film according to the invention preferably has an area of from 0.5 cm² to 10 cm², especially preferably from 2 cm² to 8 cm².

The area density of the oral thin film according to the invention is preferably at least 10 g/m², more preferably at least 20 g/m², or at least 30 g/m², or most preferably at least 50 g/m², or less than or equal to 400 g/m², more preferably less than or equal to 350 g/m², or less than or equal to 300 g/m², or most preferably less than 150 g/m². Preferably, the area density is 10 to 400 g/m², more preferably 20 to 350 g/m², or 30 to 300 g/m², most preferably 50 to 200 g/m².

Preferably, the oral thin film has a thickness of from about 10 μm to about 1000 μm, especially preferably from about 20 μm to about 500 μm.

The active pharmaceutical ingredient released from the oral thin film is preferably absorbed either at the application site, that is to say via the oral mucosa, or it is transported further and absorbed at a different site (for example in the gastrointestinal tract once the active ingredient released in the oral cavity has been swallowed).

The residence time of the oral thin film according to the invention at the application site (for example oral cavity) or the disintegration time lies preferably in the range of 10 seconds to 10 minutes, more preferably in the range of 30 seconds to 8 minutes, and most preferably in the range of 1 minute to 5 minutes.

The present invention relates to a method for producing an oral thin film as described above comprising the steps of:
a) producing a solution, dispersion or melt which at least comprises the first polymer, the second polymer and the at least one active pharmaceutical ingredient;
b) foaming the solution, dispersion or melt by introducing a gas or gas mixture, by chemical gas generation, or by expanding a dissolved gas, optionally after prior addition of a foam-stabilising agent;
c) spreading the foamed solution, dispersion or melt onto a coating substrate; and
d) solidifying the spread solution, dispersion or melt by drying and removal of the solvent or by cooling.

The term “drying” is understood to mean that solvent, especially water, is removed from the oral thin film. It is not necessary here that all solvent, such as water, is removed from the oral thin film, and instead it is sufficient if the main proportion of solvent is removed from the oral thin film, so that the foam is solidified. The oral thin film may therefore have a residual water content following the drying.

By solvent removal, the foam solidifies during the drying, with the formed voids obtaining a permanent structure. Oral thin films with desired surface dimensions or geometric shapes can be obtained in that the foamed mass is poured into corresponding moulds prior to the drying or in that the individual oral thin films are punched out from a larger two-dimensional piece.

The shape, number and size of the produced voids can be influenced by means of different method parameters, for example by the type and concentration of the polymers, by the viscosity of the polymer mass, by the control of the foaming process, by the selection of the foam-stabilising agents, etc.

Alternatively to the above-described method, it is possible to produce the oral thin film according to the invention by means of a method in which the voids within the polymer matrix are formed by introducing a hydrophobic solvent not miscible with the solvent used to produce the stated solution or dispersion.

Here, an emulsion is produced which contains the hydrophobic solvent in the form of finely distributed droplets.

By removing the solvent during the subsequent drying, droplet- or bubble-like voids remain in the polymer matrix.

Furthermore, in a modification of the above-described method, the stated voids are generated in such a way that auxiliary agents that form a gas or gases are added to the polymer- and active-ingredient-containing solution or dispersion, whereby the mass is foamed. This foaming can occur by gas development either during the production of the polymer mass or during the coating of this mass onto the substrate, or only during the subsequent drying process. Substances or substance mixtures suitable for gas formation are known to a person skilled in the art. The foaming may also be brought about by expanding a previously dissolved gas. Especially, an inert gas, such as nitrogen, carbon dioxide or helium, or a mixture thereof can be used as gas.

When producing the oral thin film according to the invention, a melt of the matrix polymer or polymer mixture can, alternatively, also be used as a starting point. The processing is performed in principle similarly to the method for hot-melt coating compounds known from the prior art.

Gas or a gas mixture is introduced into the stated polymer melt by one of the above-mentioned methods in order to foam the melt. The melt is then spread on a suitable substrate or extruded or poured into a mould and can then be left to cool or solidify. Processing from the melt is not an option if the intended active ingredient is unstable or volatile at the melting temperature of the polymer melt. If necessary, auxiliary agents can be added to the polymer melt to reduce the melting point.

According to a further modification of the production method described above, the polymer matrix is initially produced in the form of a block. From this, i.e. after drying or solidification, the desired dosage form is subsequently separated by cutting.

This means that in this embodiment the above-described steps c) and d) are replaced by the following steps c2) and d2):
c2) producing a block, starting from the solution, dispersion or melt;
d2) cutting the solidified block in order to obtain planar forms.

The present invention also relates to an oral thin film obtainable by the method described above.

In addition, the present invention relates to an oral thin film, as described above or obtainable by the above-described method, as a medicament.

The present invention additionally relates to an oral thin film, as described above or obtainable by the above-described method, wherein ketamine, preferably (S)-ketamine, or a pharmaceutically acceptable salt thereof, is used as active pharmaceutical ingredient for use in the treatment of pain and/or depression, especially to reduce the risk of suicide and/or for use as a general anaesthetic, preferably to initiate or carry out general anaesthesia, or as a supplement in the case of local anaesthesia and/or as an analgesic.

The preferred embodiments described above for the multi-layer oral thin film are also applicable for the method according to the invention, the multi-layer oral thin film obtained by this method, and use thereof as a medicament.

The invention will be explained in greater detail hereinafter on the basis of non-limiting examples.

### EXAMPLES

The following polymers were used in the following examples.
PVA 4-88: Polyvinyl alcohol (MW<100,000)
PVA 40-88: Polyvinyl alcohol (MW>100,000)
Polyox WSR N12: Polyethylene oxide polymer (MW approximately 1,000,000)
Polyox WSR 303: Poly(ethylene) oxide polymer (MW approximately 7,000,000)
Proloc 15: Mixture of polyacrylate and amylopectin (MW>100,000)

### Example 1

Foamed oral thin films with the compositions according to Tables 1 to 5 without active pharmaceutical ingredient were produced as follows.

The individual oral thin films were produced by mass production techniques known to a person skilled in the art, for example by stirring/mixing the contained components by means of a stirring motor and suitable stirring tools. Gas is injected into the resultant mass by stirring, for example by means of a foam whipping machine. The foamy mass is coated onto a coating substrate in a constant layer thickness using suitable equipment (roller applicator, squeegee, coating box, etc.). All temperature-stable web-like materials from which the dry film can be removed again can serve as coating substrates. This can be ensured by the material selection of the coating substrate (different surface tensions between foamy mass and substrate), or by suitable dehesive coatings of the coating substrate with, for example, silicones or fluoropolymers. The process solvent or solvents contained, usually water or mixtures of water and organic, water-miscible solvents, are removed by drying. The oral thin films can be provided in the appropriate size by cutting or punching from the solid foam layer thus obtained.

TABLE 1

| Ingredient | Function | Amount [wt. %] |
|---|---|---|
| PVA 4-88 | Polymer | 74.6 |
| Polyox WSR N12 | Polymer | 2.2 |
| | (disintegration | |
| | retarder) | |
| FD&C Red 40 | Colouring agent | 0.4 |
| Saccharin Na | Sweetener | 2.0 |
| Sucralose | Sweetener | 4.0 |
| Glycerol | Plasticiser | 9.0 |
| Menthol | Flavouring | 2.0 |
| Cherry Flavour | Flavouring | 6.0 |

TABLE 2

| Ingredient | Function | Amount [wt. %] |
|---|---|---|
| PVA 4-88 | Polymer | 81.1 |
| Polyox WSR 303 | Polymer | 0.5 |
| | (disintegration | |
| | retarder) | |
| FD&C Red 40 | Colouring agent | 0.4 |
| Saccharin Na | Sweetener | 2.0 |
| Sucralose | Sweetener | 4.0 |
| Glycerol | Plasticiser | 9.0 |
| Cherry Flavour | Flavouring | 6.0 |

TABLE 3

| Ingredient | Function | Amount [wt. %] |
|---|---|---|
| PVA 4-88 | Polymer | 80.6 |
| Polyox WSR 3032 | Polymer | 1.0 |
| | (disintegration | |
| | retarder) | |
| FD&C Red 40 | Colouring agent | 0.4 |
| Saccharin Na | Sweetener | 2.0 |
| Sucralose | Sweetener | 4.0 |
| Glycerol | Plasticiser | 9.0 |
| Menthol | Flavouring | 2.0 |
| Cherry Flavour | Flavouring | 6.0 |

TABLE 4

| Ingredient | Function | Amount [wt. %] |
|---|---|---|
| PVA 4-88 | Polymer | 79.6 |
| Polyox WSR 3032 | Polymer | 2.0 |
| | (disintegration | |
| | retarder) | |
| FD&C Red 40 | Colouring agent | 0.4 |
| Saccharin Na | Sweetener | 2.0 |
| Sucralose | Sweetener | 4.0 |
| Glycerol | Plasticiser | 9.0 |
| Menthol | Flavouring | 2.0 |
| Cherry Flavour | Flavouring | 6.0 |

TABLE 5

| Ingredient | Function | Amount [wt. %] |
|---|---|---|
| PVA 4-88 | Polymer | 79.6 |
| Proloc 15 | Polymer | 2.0 |
| | (disintegration | |
| | retarder) | |
| FD&C Red 40 | Colouring agent | 0.4 |
| Saccharin Na | Sweetener | 2.0 |
| Sucralose | Sweetener | 4.0 |
| Glycerol | Plasticiser | 9.0 |
| Menthol | Flavouring | 2.0 |
| Cherry Flavour | Flavouring | 6.0 |

The oral thin films with the compositions according to Tables 1 to 6 had much longer disintegration times than compositions that contained no polymer with a MW greater than 100,000 g/mol.

### Example 2

Foamed oral thin films with the compositions according to Tables 6 and 7 with (S)-ketamine HCl as active pharmaceutical ingredient were produced as described above.

TABLE 6

| | | Amount | |
|---|---|---|---|
| | | per dose | Amount |
| Ingredient | Function | [m/1.67 cm³) | [wt. %] |
| (S)-ketamine HCl | Active ingredient | 16.1 | 55.00 |
| PVA 4-88 | Polymer | 5.1 | 17.25 |
| PVA 40-88 | Polymer | 5.1 | 17.25 |
| | (disintegration | | |
| | retarder) | | |
| Saccharin Na | Sweetener | 0.3 | 1.00 |
| Sucralose | Sweetener | 0.6 | 2.00 |
| Glycerol | Plasticiser | 1.3 | 4.50 |
| Flavour PHL-131985 | Flavouring | 0.3 | 1.00 |
| Flavour PHL-097336 | Flavouring | 0.6 | 2.00 |

| | |
|---|---|
| Disintegration time | 31 sec |

TABLE 7

| | | Amount | |
|---|---|---|---|
| | | per dose | Amount |
| Ingredient | Function | [m/1.67 cm³) | [wt. %] |
| (S)-ketamine HCl | Active ingredient | 16.1 | 50.00 |
| PVA 4-88 | Polymer | 12.8 | 39.50 |
| Saccharin Na | Sweetener | 0.3 | 1.00 |
| Sucralose | Sweetener | 0.6 | 2.00 |
| Glycerol | Plasticiser | 1.4 | 4.50 |
| Cherry Flavour | Flavouring | 1.0 | 3.00 |

| | |
|---|---|
| Disintegration time | approximately 9 sec |

The oral thin film with the composition according to Table 6 had a much longer disintegration time than the oral thin film with the composition according to Table 7 that contained no polymer with a MW greater than 100,000 g/mol.

In addition, the release rates of the active ingredient were determined for the foamed oral thin film with the composition according to Table 6.

Release Measurement:

With the in vitro release, S-ketamine is released from S-ketamine HCl oral thin films (OTF) and is determined. The active ingredient is released in phosphate buffer pH 6.8 USP and is then determined by a gradient reverse phase HPLC method. The quantification was performed against an external standard.

The release is performed with Dissolution Apparatus 2—(Paddle over sinker) according to USP <711>.

TABLE 8

| | |
|---|---|
| Sinker: | Stainless Steel Capsule Sinker |
| | with 10 Spirals, 31.0 × |
| | 11.0 mm Capacity (Sotax Style) |
| Stirring speed: | 50 rpm |
| Distance between the vessel base and | 25 mm ± 2 mm |
| the lower edge of the paddle: | |
| Temperature: | 37° C. ± 0.5° C. |
| Release medium: | Phosphate buffer pH 6.8 USP |
| Release medium volume: | 900 mL |
| Sample measurement times: | 1, 3, 5, 10 and 15 min |
| Sample volume: | 5.0 mL |

The determined release rates are summarised in Table 9.

TABLE 9

| In-vitro release | | [% of LC*] |
|---|---|---|
| after 1 min | Mean | 57 |
| | Min | 52 |
| | Max | 59 |
| | RSD [%] | 5.2 |
| after 3 min | Mean | 101 |
| | Min | 97 |
| | Max | 105 |
| | RSD [%] | 2.8 |
| after 5 min | Mean | 104 |
| | Min | 101 |
| | Max | 110 |
| | RSD [%] | 2.9 |
| after 10 min | Mean | 104 |
| | Min | 102 |
| | Max | 108 |
| | RSD [%] | 2.3 |
| after 15 min | Mean | 104 |
| | Min | 101 |
| | Max | 109 |
| | RSD [%] | 2.7 |

*Label Claim = specified content

The active ingredient release from an oral thin film according to Table 7 (fast-release) was compared with the active ingredient release from an oral thin film distinguished by a especially slower active ingredient release (slow-release).

The composition of the oral thin film with slow active ingredient release is specified in Table 10.

TABLE 10

| | | Amount |
|---|---|---|
| Ingredient | Function | [wt. %] |
| (S)-ketamine HCl | Active ingredient | 30.00 |
| Proloc 15 | Polymer | 10.00 |
| PVA 40-88 | Polymer | 49.50 |
| | (disintegration | |
| | retarder) | |
| Saccharin Na | Sweetener | 1.00 |
| Sucralose | Sweetener | 2.00 |
| Glycerol | Plasticiser | 4.50 |
| Cherry Flavour | Flavouring | 3.00 |
| Area density | | 152.9 g/m² |
| (dry) | | |
| Area density | | 156.0 g/m² |
| (incl. res. solvent) | | |

In addition, the release rates of the active ingredient, as described above, were determined for the foamed oral thin film with the compositions according to Table 7 and Table 10.

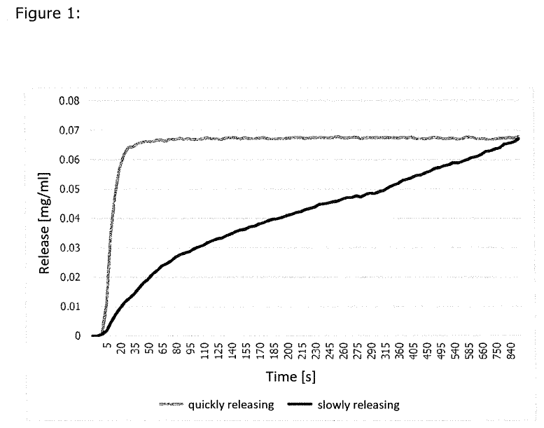

The determined release rates are shown in FIG. 1.

### Example 3

A foamed oral thin film with the composition according to Table 11 with (S)-ketamine HCl as active ingredient were produced as described above.

TABLE 11

| | | Amount | |
|---|---|---|---|
| | | per dose | Amount |
| Ingredient | Function | [mg/2.72 cm³) | [wt. %] |
| (S)-ketamine HCl | Active ingredient | 16.1 | 50.00 |
| PVA 4-88 | Polymer | 12.8 | 39.50 |
| Saccharin Na | Sweetener | 0.3 | 1.00 |
| Sucralose | Sweetener | 0.6 | 2.00 |
| Glycerol | Plasticiser | 1.5 | 4.50 |
| Masking flavour | Flavouring | 0.3 | 1.00 |
| Raspberry flavour | Flavouring | 0.6 | 2.00 |

| | |
|---|---|
| Disintegration time | approximately 3 sec |

In addition, the release rate of the active ingredient, as described above, was determined for the foamed oral thin film with the composition according to Table 11.

The determined release rates are summarised in Table 12.

TABLE 12

| In-vitro release | | [% of LC*] |
|---|---|---|
| after 1 min | Mean | 100 |
| | Min | 93 |
| | Max | 106 |
| | RSD [%] | 5.0 |
| after 3 min | Mean | 104 |
| | Min | 100 |
| | Max | 109 |
| | RSD [%] | 3.4 |
| after 5 min | Mean | 105 |
| | Min | 100 |
| | Max | 109 |
| | RSD [%] | 3.6 |
| after 10 min | Mean | 105 |
| | Min | 100 |
| | Max | 110 |
| | RSD [%] | 3.8 |
| after 15 min | Mean | 105 |
| | Min | 101 |
| | Max | 109 |
| | RSD [%] | 3.6 |

*Label Claim = specified content

1. An oral thin film comprising a polymer matrix in the form of a solidified foam having voids, wherein the polymer matrix contains at least one active pharmaceutical ingredient, at least one first polymer which comprises a water-soluble polymer and at least one second polymer which comprises a mucoadhesive polymer having a number-average molecular weight of equal to or greater than 100,000 g/mol.

2. The oral thin film according to claim 1, characterised in that the at least one mucoadhesive polymer comprises a water-soluble polymer.

3. The oral thin film according to claim 1, characterised in that the first polymer comprises a polymer having a number-average molecular weight of less than 100,000 g/mol.

4. The oral thin film according to claim 1, characterised in that the first polymer is selected from the group comprising starch and starch derivatives, dextrans, cellulose derivatives, such as carboxymethyl cellulose, hydroxypropyl cellulose, hydroxyethyl cellulose, hydroxypropyl methylcellulose, hydroxypropyl ethyl cellulose, sodium carboxymethyl cellulose, ethyl or propyl cellulose, polyacrylic acids, polyacrylates, polyvinylpyrrolidones, vinyl pyrrolidone/vinyl acetate copolymers, polyvinyl alcohols, polyethylene oxide polymers, polyacrylamides, polyethylene glycols, gelatines, collagen, alginates, pectin, pullulan, tragacanth, chitosan, alginic acid, arabinogalactan, galactomannan, agar, agarose, carrageenan, and natural gums.

5. The oral thin film according to claim 1, characterised in that the second polymer is selected from the group comprising polyethylene oxide polymers, starch derivatives, polyacrylic acids, amylopectins and/or polyvinyl alcohols, polyacrylates or polyacrylic acids and amylopectins.

6. The oral thin film according to claim 1, characterised in that the first polymer is contained in the oral thin film in an amount of 40 to 95 wt. % in relation to the total weight of the oral thin film.

7. The oral thin film according to claim 1, characterised in that the second polymer is contained in the oral thin film in an amount of 0.1 to 20 wt. % in relation to the total weight of the oral thin film.

8. The oral thin film according to claim 1, characterised in that the at least one active pharmaceutical ingredient is contained in the oral thin film in an amount of 10 to 60 wt. % in relation to the total weight of the oral thin film.

9. The oral thin film according to claim 1, characterised in that the voids are isolated from one another and are present in the form of bubbles, with air or a gas.

10. The oral thin film according to claim 1, characterised in that the voids are connected to one another and preferably form a channel system penetrating the polymer matrix.

11. The oral thin film according to claim 1, characterised in that said voids account for a volume fraction of from 5 to 98%, in relation to the total volume of the oral thin film.

12. A method for producing an oral thin film according to claim 1, characterised by the steps of:
a) producing a solution, dispersion or melt which at least comprises the first polymer, the second polymer and the at least one active pharmaceutical ingredient;
b) foaming the solution, dispersion or melt by introducing a gas or gas mixture, by chemical gas generation, or by expanding a dissolved gas, optionally after prior addition of a foam-stabilising agent;
c) spreading the foamed solution, dispersion or melt onto a coating substrate; and
d) solidifying the spread solution, dispersion or melt by drying and removal of the solvent or by cooling.

13. The method according to claim 12, characterised in that steps c) and d) are replaced by the following steps c2) and d2):
c2) producing a block, starting from the solution, dispersion or melt;
d2) cutting the solidified block in order to obtain planar forms.

14. An oral thin film obtainable by a method according to claim 12.

15. (canceled)

16. A method of administering a medicament comprising providing the oral thin film of claim 1 to a subject.

17. The oral thin film according to claim 1, characterised in that the second polymer is selected from polyacrylates or polyacrylic acids and amylopectins and mixtures thereof.

18. The oral thin film according to claim 9, characterised in that the bubbles are filled with an inert gas.

19. The oral thin film according to claim 18, characterised in that the inert gas is nitrogen, carbon dioxide, helium or a mixture of at least two of these gases.

20. The oral thin film according to claim 11, characterised in that said voids account for a volume fraction of from 50 to 80%, in relation to the total volume of the oral thin film.
